(19) United States
(12) Patent Application Publication
(10) Pub. No.: US 2024/0392364 A1
(43) Pub. Date: Nov. 28, 2024

(54) SOLUTION FOR THE PRESERVATION OF CELLS

(71) Applicant: ANACYTE LABORATORIES GMBH, Hamburg (DE)

(72) Inventor: HAUKE SIMON GÜNTHER, Hamburg (DE)

(21) Appl. No.: 18/689,552

(22) PCT Filed: Sep. 5, 2022
(86) PCT No.: PCT/EP2022/074652

§ 371 Date: Mar. 6, 2024

(30) Foreign Application Priority Data
Sep. 6, 2021 (EP) 21195103.3

(51) Int. Cl. C12Q 1/6869; C12Q 1/6806

(52) U.S. Cl. CPC C12Q 1/6869; C12Q 1/6806; C12Q 2600/158

(57) ABSTRACT

The invention refers to a solution for the conservation or preservation of cells in blood samples or other body fluids, in cell culture or tissue samples. The solution contains a cell membrane permeable proton carrier and is essentially free of chaotropic substances, alcohols and detergents. Furthermore, the solution according to the present invention has a pH value which produces an intracellular pH of the cells of less than 6. Such a solution is advantageously used in sample tubes, blood collection syringes or blood collection tubes.

The present invention generally relates to the stabilization of blood samples and in particular to the preservation of cells, most notably circulating tumor cells, in blood samples or other body fluids. The invention relates in particular to a preservative solution and a use for the rapid, permanent interruption of the metabolism of cells to stabilize the gene expression state, in particular the transcriptome while preserving the morphology, which enables subsequent molecular analysis of the transcriptome. Further, the invention relates to the use of preservative solutions and sample tubes or blood collection tubes or syringes in which a solution according to the present invention is provided.

### BACKGROUND OF THE INVENTION

Biomolecules are subject to constant turnover in an intact, metabolizing cell. Depending on e.g. environmental conditions or the stage of differentiation or other circumstances having an influence on metabolism, this turnover of molecules takes place through new synthesis and degradation. A change in biomolecule composition occurs within cells in a specific, controlled and targeted manner. Outside cells, on the other hand, biomolecules are subject to unspecific, uncontrolled and random degradation. In order to understand the biological functions of a molecule in a cell, it is crucial to understand its function in the context of the other biomolecules present at the same time. Single cell transcriptome analysis (RNASeq) in particular has recently made it clear that cells of the same type can exhibit very different gene expression patterns. In particular, the analysis of tumor cells has shown that a solid tumor can be heterogeneous in its gene expression. Therefore, analyzing the transcriptome of individual cells is of general interest.

Analyzing the gene expression status of a cell is complicated by various circumstances. These include the instability of many biomolecules, especially RNA, when they are present outside of cells. However, the storage of intact cells until they are analyzed can also have a considerable influence on gene expression. It is common practice, for example, to temporarily store cells obtained from the cell culture at 4° C. during harvesting, to remove medium and growth factors and not only to induce considerable temperature changes by repeated centrifugation for preparation, but also to oscillate cell densities as a result. It is known that cell density has a considerable influence on differentiation and cell division. The same applies after removal of tissue or tumors. Preparing changes the nutrient supply, oxygen and CO₂ partial pressures and the microenvironment. This is followed by a rapid response from the cells in the form of changed expression patterns, which are most quickly noticeable at the transcriptome level. It is therefore important to preserve the transcriptome as far as possible in the respective present condition and to protect it from manipulation-related changes.

The difficulties of transcriptome analysis become clear by the example of biobanks. Human tumor samples that are given to biobanking after an operation, for example, are usually fixed with 10% formaldehyde immediately after removal. However, this has the considerable disadvantage that the transcriptome, which is important for analyzing the actual state, is cross-linked. This makes molecular analysis considerably more difficult and it is generally no longer possible to isolate intact molecules. For this reason, the fresh samples are usually stored at room temperature until they can be further processed by qualified staff. Further processing is usually carried out by professionally dividing the sample in order to freeze one part for subsequent molecular analyses and to keep another part ready for morphological analyses after formaldehyde fixation. The storage period between collection and further processing can be several hours under certain circumstances and holds considerable risks for the validity of the biobanks or the preserved samples. Further, subsequent thawing of the sample for molecular analyses leads to induction of RNA degradation processes and thus to changes in the transcriptome.

In tumor biology, a great heterogeneity of the molecular composition of cells within a tumor has been demonstrated in recent years. In this context, circulating tumor cells (CTC) are of particular importance, as an analysis of these cells makes it possible to identify therapy options and to monitor the course of the disease and therapy. Circulating tumor cells are cells that have shed from a primary tumor. They can either be present in the lymphatic system or circulate in the bloodstream. With at least some types of circulating tumor cells, there is a possibility that they will colonize other organs or tissues and form new tumors or metastases. The majority of cancer-related deaths are not due to primary tumors, but to metastases and secondary tumors derived therefrom.

Diagnostic methods that enable analysis of CTCs can contribute significantly to the selection of personalized and effective treatment methods. Furthermore, analyzing CTCs can be helpful in establishing a prognosis for the patient at an early stage, as it is has been recognized that these circulating cells already exist and can be detected at an early stage of the disease. Analyses of CTCs are also very important for examining the efficacy of medicaments.

However, CTCs are only present in very small quantities in the respective body fluids and are also sensitive to manipulation during analyses. As mentioned above, all biomolecules in an intact, metabolizing cell are also subject to constant turnover. Depending on various influencing factors, for example environmental conditions or the stage of differentiation or other circumstances affecting metabolism, this turnover of molecules takes place through new synthesis and degradation. A change in biomolecular composition occurs within cells in a specific, controlled and targeted manner. In order to understand the biological functions of a molecule in a cell, it is crucial to observe the respective function in the context of the other biomolecules present at the same time. This requires the preservation of cells and the stabilization of their metabolic and morphological state.

Although the existence of CTCs has been known for a long time, it was only a few years ago that they came to the fore of research in order to improve treatment options for tumor patients. Some methods of preserving blood have been standard worldwide for decades. EDTA, citrate or heparin additives prevent blood clotting and allow the components of the blood to be analyzed. However, CTCs cannot be stabilized very well with the help of these additives, as many normal blood functions remain intact even after blood collection, i.e. ex vivo. For example, the immune system reacts and attempts to eliminate CTCs, or CTCs that have already been attacked by chemotherapy undergo necrosis or apoptosis.

Storage time between collection and further processing of blood samples can also be many hours and harbors considerable additional risks. The expression status of the CTCs changes with temperature and environmental conditions, to which the CTCs react with changed gene expression. This leads to an undesirable change in the molecular image in the subsequent analysis.

It is also known that even normal blood cells are subject to considerable changes due to storage. On the other hand, the storage of blood is part of the diagnostic routine, as certain procedures and times are specified in everyday hospital life. Storage-related changes include not only a change in the expression status of blood cells, but also, for example, a kind of disintegration of cells, which induces so-called debris, i.e. aggregates of cell material lying around, which not only make the analysis of cells more difficult, but can also result in invalid analyses (see also U.S. Pat. No. 7,863,012 B2). This shows that not only the preservation of cellular integrity, but also the preservation of the expression status of cells over a longer period of time is of decisive importance for the analysis of CTCs. RNA stability is particularly important, as the transcriptome can provide crucial insights into tumor biology and therapeutic options.

“Molecular analysis of the transcriptome” refers to the analysis of cellular RNA using molecular biological methods. These include, for example, spectroscopic quantification, Northern hybridization, reverse transcription with or without hybridization of a biochip or also the amplification of individual transcripts by polymerase chain reaction and RNA-seq methods (total RNA, mRNA, amplicon sequencing). Such molecular biological analysis methods are generally known and are not subject of the present invention. “Morphological analysis” as used herein means the analysis of single or multiple cells up to the analysis of a cell cluster in the natural context or natural environment and refers to size, shape, granularity, etc. The term “morphometric analysis” as used herein means the analysis of one or more cellular characteristics of individual or several cells, e.g. the analysis of the expression of a specific cell marker.

### PRIOR ART

Various ways of maintaining cellular integrity have been proposed in the literature. As a rule, these are based on cross-linking agents such as formaldehyde, paraformaldehyde and glutaraldehyde (e.g. EP 0 214 613 A2, DE 40 39 716 A1). U.S. Pat. No. 4,971,783 A describes a process for preparing tissue. US patent U.S. Pat. No. 5,976,829 A describes a formaldehyde-based fixative that is also suitable for DNA/RNA analysis.

Another approach is mentioned in citations, according to which methylol derivatives enable the fixation of blood components. Methylol is also used as a preservative in the cosmetics industry. The exact mechanism of action is unknown, but formaldehyde appears to play an important role. However, the cross-linking nature of these fixative solutions, in particular the cross-linking of nucleic acids, makes it difficult to analyze cells at molecular level. U.S. Pat. No. 5,976,829 A, among others, attempts to solve this problem by a fixative comprising an aldehyde, an alcohol, a chelating agent and a buffer that does not contain amino groups.

In the scientific field, other methods are also used for the fixation of cells or molecules. These include, for example, freezing at low temperatures, using alcohol-based fixatives (e.g. methanol, ethanol, glycol) or chaotropic reagents (e.g. isothiocyanate), which enable stabilization of molecules while destroying morphological contexts.

Various solutions are also available in the prior art for fixating tissues without cross-linking while simultaneously retaining their structure. EP 2 126 542 B1, FR 2 852 392 A1, WO 2013/131816 A1 and WO 03/029783 A1 describe mixtures for tissue fixation using organic solvents. Solutions are also available to stabilize biomolecules without preserving the morphological context. Non-crosslinking methods for the preservation of cells and tissue are usually based on dehydration by incubation in organic solutions such as alcohol or acetone preferably together with strong acids, e.g. a combination of methanol/glacial acetic acid. Nucleic acids are comparatively well protected. Acetone/glacial acetic acid fixes somewhat more mildly in comparison. However, it is not possible to analyze molecules after alcohol or acetone fixation at the cellular level without rehydration. Nucleic acids, in particular RNA, are exposed to unhindered degradation due to dissolution of the compartmentalization, i.e. damage to the cellular membrane systems (e.g. lysosomes) through rehydration.

Other publications describe further solutions for fixating cells using various mechanisms. For example, a corresponding solution disclosed in WO 2012/150479 A1 contains halogen cyano acetamides and US 2015/0050689 A1 describes a combination of polyamines and acids that react to release aldehydes, in particular formaldehyde.

In practice, biological preparations are usually deep-frozen to obtain RNA. To obtain RNA, the samples are then lysed in frozen state under strongly denaturing or chaotropic conditions. Although the RNA isolated in this way is often of satisfactory quality, it is not possible to qualify the cell(s) desired for examination by morphological or morphometric analysis, although molecular analysis of a subpopulation is particularly desirable in practice. However, morphological or morphometric selection of such a subset prior to freezing requires cross-linking formaldehyde fixation to maintain the expression status, which makes molecular analysis difficult or impossible. The alternative of non-crosslinking, dehydrating fixation to isolate a subset of cells is also unfavorable, as the subsequent selection of a subpopulation in the rehydrated state leads to rapid degradation of the RNA.

CN202011616524 teaches the stabilization of DNA in whole blood using a complex mixture of anticoagulants and stabilizers at neutral or near-neutral pH.

Another proposal according to the prior art for non-crosslinking fixation of cells and tissue is Hepes mediated glutamic acid protection (DE10021390C2). Here, an amino acid-containing solution is proposed to morphologically preserve tissue by dehydrating paraffin embedding as long-term preservation. Although the dehydrating fixation is carried out comparatively mild by using acetone, there are structural changes in the tissue due to the lack of cross-linking. The system is comparatively ineffective in stabilizing the transcriptome, although processing is carried out at low temperature.

Ringwald et al (Transfusion Medicine Reviews, 20 (2), 2006) stabilize platelets with a mixture of different salts and acids. In particular, acetic acid is used to maintain platelet metabolism at neutral pH during storage for transfusion purposes. As nucleus-free “cells”, platelets are transcriptionally inactive.

In a recently published method for RNAseq transcriptome analyses, cells are fixed with a mixture of water, methanol, acetic acid and glycerol (ACME). This method changes cell structure and morphology and it cannot be ruled out that the alcoholic reagents used will release RNA molecules and protein markers from the cells. In addition, rehydration results in degradation of RNA transcripts. The teaching of PCT/EP2015/061678 (WO 2015/181220 A1) follows a similar approach.

Various chaotropic reagents are commercially available for stabilizing RNA while destroying morphological relationships (e.g. RNAlater, Ambion; ProtectAll, Qiagen).

A modification without organic solvent but using bisulphites is described in U.S. Pat. No. 5,432,056 A. Here, a bisulphite-containing acidic system is proposed for fixating tissue sections or other thin-layered samples. U.S. Pat. No. 6,337,189 B1 in turn describes a urea compound in combination with alcohol for non-crosslinking fixation of cytological preparations. However, the morphological preservation of cellular structures is limited in all these methods.

Such methods are unsuitable for the preservation of whole blood for the analysis of CTCs, as they are either not suitable for routine diagnostics, destroy cellular integrity or do not guarantee effective molecule stabilization.

However, U.S. Pat. No. 10,091,984 B2 teaches that a formaldehyde-releasing urea compound is capable of morphologically stabilizing CTCs when the formaldehyde is simultaneously scavenged by glycine. Even if this disclosure does not postulate a covalent modification of the CTC molecules, the role of formaldehyde is unclear.

The prior art has limitations with regard to the possible solutions for preserving the transcriptome of intact cells in the status quo in order to carry out a molecular analysis at a later point in time. In particular, the prior art has limitations when molecular analysis of the transcriptome of specific cells is to be performed: (A) Cross-linking agents preserve the expression status relatively slowly depending on the diffusion rate of the agent and complicate molecular analysis; (B) Non-cross-linking fixation by organic solutions requires rehydration and forces the change of the transcriptome by uncontrolled molecular degradation; (C) Lysis of a cell assembly by chaotropic reagents does not allow differentiation of individual cells. In theory, the chaotropic lysis of individual cells is possible. However, isolation would be preceded by the destruction of their context, which would lead to a change in the transcriptome.

Therefore, the problem to be solved was to develop a solution that makes it possible to interrupt the metabolism in cells of a culture or tissue at a time to be determined by the experimenter, in particular to stabilize the transcriptome at the present time and at the same time leave cells and cell compartmentation intact in order to prevent uncontrolled degradation of the RNA. The solution is intended to enable time-delayed transcriptome analysis for the preservation or performance of cell-qualifying morphological or morphometric analyses.

The problem to be solved also consisted of developing a method and suitable means that make it possible to interrupt the metabolism or biomolecule turnover in blood cells, in particular of CTCs or other body fluids, in order to ensure a temporary, highly stabilized, non-cross-linked preservation of the actual state of the genome, transcriptome and proteome and to prevent uncontrolled degradation of the molecules, while at the same time keeping the cells morphologically intact and preventing coagulation of the blood. The solution to the problem should enable time-delayed processing for the isolation of CTCs and the performance of molecular and possibly morphological analyses of the CTCs or other cells.

### SUMMARY OF THE INVENTION

The aforementioned problems are solved by the present invention. A first aspect of the present invention is a solution for preserving cells, in particular at least one eukaryotic cell and in particular tumor cells and circulating tumor cells in a blood sample or another body fluid, cells from cell culture or cells in the tissue, which is characterized by
a) containing a membrane-permeable proton carrier,
b) being substantially free from chaotropic substances, alcohols and detergents, and
c) having a pH value that produces an intracellular pH of the cell or cells of less than 6.

Furthermore, another aspect of the present invention is in the use of such a solution according to the first aspect of the invention for conserving, preserving or/and fixating cells, in particular at least one eukaryotic cell, in particular tumor cells or circulating tumor cells in a blood sample or another body fluid or cells from cell culture or cells in the tissue while largely preserving the cell morphology, in particular for a molecular analysis of the genome, the transcriptome and the proteome.

A third aspect of the present invention relates to sample tubes, blood collection syringes or blood collection tubes containing a solution according to the first aspect of the invention.

Advantages and details of the present invention are apparent from the claims, the following detailed description and the embodiment.

### DETAILED DESCRIPTION OF THE INVENTION

Intact cell membranes are not permeable for passive proton transport and proton transport across the cell membrane is highly controlled. The present invention is based on the finding that an abrupt and rapid change (lowering) of the pH value within the cells leads to a loss of function of the cellular machinery for the synthesis and degradation of molecules and, via the inactivation of enzymatic activity, to a halt in metabolism without interfering with cellular compartmentalization. The essential characteristic of the solution according to the present invention is therefore that the metabolic processes are shut down very quickly by reducing the pH value, thus avoiding the environment-dependent change in the expression state.

According to the present invention, this change in the intracellular pH is achieved by membrane-permeable proton carriers contained in the solution. A proton carrier is understood to be a molecule which is capable of overcoming the cell membrane, preferably in a passive way, in protonated form under suitable conditions and which decomposes into anion and proton within the cell. Suitable conditions are created, for example, by mixing cells with a proton carrier with a pKs in the acidic range in an acidic environment. The protonated form of the proton carrier passes through the cell membrane and dissociates within the cell due to the higher intracellular pH. As an ion, it is not possible for the proton carrier to leave the cell, so that at equilibrium the intracellular pH is determined by the extracellular pH.

Furthermore, the present invention largely neutralizes the charge of nucleic acids. This, in particular, in the case of RNA leads to partial precipitation from the aqueous environment and thus to stabilization against both enzymatic and alkaline hydrolysis.

In the context of the present invention, and in particular for the preservation of the morphology of the cells, it is furthermore essential that chaotropic substances, which are present in many corresponding preservative solutions in the prior art, are essentially completely absent. Chaotropic substances such as perchlorates such as sodium perchlorate, thiocyanates such as guanidium thiocyanate, but also guanidinium hydrochloride and barium salts are substances that dissolve ordered hydrogen bonds in water, break up the water structure and cause an increase in entropy. They interfere with the hydration shell of biomolecules, leading to their denaturation and, if present in appropriate concentrations, to the complete dissolution of cells.

In the context of the present invention, the term “substantially complete absence” means that a substance can only be present in such a maximum amount that does not destroy the morphological integrity of the cells. In preferred embodiments, the solution according to the invention is completely free of chaotropic substances.

Analogous to the absence of chaotropic substances, the solution according to the present invention is also characterized by an essentially complete absence of alcohols and detergents. In preferred embodiments, these substances are also completely excluded from the solution according to the present invention.

Finally, the solution according to the present invention is further characterized in that it has a pH value which produces an intracellular pH of the cell or cells of less than 6.

Advantageously, membrane-permeable carboxylic acids are used as proton carriers in the context of the present invention, in particular C₂ to C₅ carboxylic acids. As already mentioned above, the present invention is characterized by the rapid arrest of metabolism while preserving the morphology of cells, in particular of cells in the blood. Cells in the blood are understood to be all cells containing cell nuclei. This metabolic arrest is also referred to below as fixation or preservation, although the mechanism of action of the present invention is different from the known cross-linking, dehydrating or denaturing fixations. The metabolism stop is preferably achieved by a dissolved carboxylic acid in combination with a low pH. The protonated form of the carboxylic acid passes through the cell membrane and dissociates inside the cell. However, other acids that are membrane-permeable in protonated form can also be used.

The acids are preferably present in the solution according to the present invention in the form of a carboxylic acid buffer system or an acid/base system, which forms and stabilizes a pH in the desired acidic range. These buffer systems preferably comprise weak acids and their corresponding bases. Blood has a very high buffering capacity. According to the invention, this buffer capacity is titrated out with the buffer system contained in the solution in order to create an acidic environment in the mixture and in particular within the cells, which in turn largely shuts down metabolic activities. According to the present invention, a buffer system which provides a sufficient amount of protonated carboxylic acid for membrane passage and at the same time releases sufficient H+ ions intracellularly is suitable.

Depending on the test material used, it may be necessary to additionally add a strong acid to the solution according to the invention to adjust the intracellular pH value so that the buffer capacity of the test material is eliminated. In particular hydrochloric acid or other mineral acids are suitable strong acids. However, these acids are not membrane-permeable and therefore in the context of the present invention do not represent proton carriers.

In particular, the proton carrier is selected and the pH of the solution according to the invention is adjusted in such a way that, after mixing with cells, the blood sample or another body fluid or tissue sample, a mixture having a pH of between 4 and 6 is obtained. The skilled person can easily make the appropriate adjustment, as he is familiar with the pH value and the buffer capacity of cell culture medium, blood samples or other body fluids or tissue samples that may be the subject-matter of interest and can therefore preset the pH value of the solution according to the desired mixing ratio. The usual mixing ratios of blood samples to be analyzed and preservation solutions are also known to the skilled person and are, for example, 1:10 for citrated blood. The mixing ratios can be adapted accordingly for other subject-matters of interest and are generally in the same range.

In the context of the present invention, acetic acid in particular is used as the carboxylic acid. Acetic acid is then preferably present in the solution according to the present invention as an acetic acid/acetate system and the pH of the solution is adjusted so that it is in any case below 6. In further preferred embodiments, the pH value and the buffer capacity of the solution according to the present invention are selected such that the resulting pH value after mixing with the sample is below 6. In particular, however, the pH value of the mixture is even between 2 and 6, more preferably between 4.8 and 5.8 and particularly preferably between 5.0 and 5.5, or between 5.1 and 5.3. Solutions according to the present invention whose pH value is below 6, preferably below 5.8 and in particular equal to or below 5.3 are particularly suitable for this purpose. Depending on the intended mixing ratio and the body fluid to be preserved, such solutions according to the present invention have a pH value in the acidic range, in particular between 1 and 5 and preferably between 1.8 and 4.5, and, for example, if intended for mixing with blood in a ratio of 1:5, preferably between 2 and 3. The information on the pH value relates to a temperature of 20° C. in the context of the present invention. As already explained above, the intracellular pH value is also set based on the pH of the solution in which the cells are present, and the buffer capacity of the test material.

The acid/base system of the proton carrier of the present invention is usually present in a concentration of 1 to 300 mM, depending on whether it is a concentrated solution which is subjected to dilution by the blood sample and other aqueous solutions, or whether it is a ready-to-use solution which is added in excess, e.g. with 10-fold volume to isolated cells. The solution according to the present invention is characterized by the possibility of being configured as a (multiple) concentrate in order to fix blood without excessive dilution. In particular, a solution according to the present invention is envisaged in which the acid/base system of the proton carrier and its anticoagulant components are configured as a 2 to 20-fold, preferably 5 to 10-fold concentrate for the preservation of blood or another body fluid in order to achieve the desired intracellular pH as described above.

In preferred embodiments of the present invention, the acid/base system and preferably an acetic acid/acetate system is present in a concentrated solution according to the invention in a concentration which is 10 mM to 300 mM, preferably 25 mM to 200 mM and particularly preferably 50 mM to 150 mM. In particular for the ready-to-use solution, on the other hand, it is preferred that the solution comprises the acid/base system in a concentration of 1 mM to 100 mM, preferably 5 mM to 50 mM and particularly preferably 10 mM to 20 mM.

In addition to the aforementioned or other proton carriers, the solution according to the invention may also comprise additional substances, such as in particular buffer substances or conventional auxiliary substances. In particular, the solution according to the present invention may contain imidazole and/or dimethyl sulphoxide (DMSO). Imidazole has a slightly acidic pKs and can cross the cell membrane in protonated or non-protonated form. Imidazole can initially support the transport of H⁺ ions into the cell interior. A suitable concentration of imidazole is, for example, 50 mM. To use the solution according to the present invention for the frozen storage of test objects, it preferably contains DMSO in a final concentration of 5 to 15%, particularly preferably 10%.

Other suitable proton carriers that can be used as a component of the preservative solution in the context of the invention are cyclic peptides such as valinomycin or nigericin. In addition, the solution according to the invention can contain further ions, in particular Cl⁻ ions.

The solution according to the present invention can also contain other substances that contribute to preservation. For example, the addition of β-mercaptoethanol or dithiotreitol or similar reagents can additionally reduce the activity of RNases. It is self-evident to the skilled person that the addition of other enzyme inhibitors, for example phosphatase inhibitors, can also have a positive influence on the preservative effect of the solution according to the invention.

In further preferred embodiments, the solution according to the present invention additionally comprises at least one amino acid. In particular, this is an amino acid naturally occurring in the tissue or in the cells. The buffer system of the present invention utilizes the protective effect of amino acids. This effect is particularly effective when such amino acids are present in a concentration of 0.1 M to 1M in total or in each case, preferably in a total concentration of 100 mM to 300 mM. Preferably, the amino acids are one or more from the group comprising glycine, alanine, proline, serine, threonine, glutamic acid and aspartic acid.

A further embodiment of the present invention is the combination of rapid pH-dependent fixation with an additional preservative active ingredient from the group of formaldehyde donors. Such active ingredients can be, for example, diazolidinyl urea or imidazolidinyl urea.

Preferably, the composition of the solution according to the present invention is also such that the osmolarity does not exceed 1 osmol. Preferably, the osmolarity of the solution is above 100 and below 500 mosmol, preferably between 250 and 350 mosmol. The solution is particularly preferably isotonic or forms an isotonic environment for the cell or cells to be preserved.

In some embodiments of the present invention, the formulation of the solution according to the invention does not completely prevent coagulation of the blood. It is known to the person skilled in the art that these coagulation phenomena can be prevented by adding complexing agents, coagulation-preventing agents or inhibiting antibodies. The presence of such complexing agents, anticoagulants or antibodies in the solution according to the present invention therefore results in further preferred embodiments of the invention. In preferred embodiments, MgSO₄, dabigatran or/and plasminogen activator (t-PA) are present as anticoagulants in the solution according to the present invention.

A further aspect of the present invention is the use of the solution according to the invention for conserving, preserving or/and fixating cells, in particular at least one eukaryotic cell, in particular tumor cells and circulating tumor cells in a blood sample or other body fluid, cells from cell culture or cells in tissue, preferably with subsequent or time-delayed molecular analysis.

This use according to the present invention is characterized in that the solution is mixed with the blood sample or other body fluid, the cells from cell culture in a corresponding medium or with cell-containing tissue, preferably directly after collection.

The molecular analysis may relate to any substance or group of substances present in the cell, in particular the genome, the transcriptome and the proteome. Preferably, a transcriptome analysis is carried out as molecular analysis on the at least one cell after treatment with the solution according to the invention.

A particular feature of the present invention is the possibility of qualifying largely intact cells on the basis of a characteristic feature and using this qualification to carry out molecular analyses. Preferably, a mixture of different cells treated with the solution according to the invention is analyzed for a marker. Preferably, this is a morphological marker (e.g. analyzed by FSC/SSC in flow cytometry) or a morphometric marker (e.g. a labelled antibody for binding to a surface protein). Thereby the mixture can consist of two, three or more different cell populations. Preferably, this marker is used to qualify at least one cell for the analysis of the cellular components, particularly preferably the transcriptome.

It may be preferred to add at least one enzyme for tissue digestion to the solution according to the invention in order to release individual cells from a tissue structure.

Preferably, the at least one enzyme is a collagenase, a dispase, or a combination thereof.

It may be preferable to store the at least one cell treated with the solution according to the invention. It may be preferred to store it at 2-8° C. Alternatively, it may be preferred to carry out this storage in a frozen state.

The use according to the invention preferably comprises at least: an input step a) treatment of cells or tissue with a solution suitable for the use and an output step f) molecular analysis of the transcriptome of at least one cell.

Preferably, the use comprises further steps which can be carried out individually or in combination between the input step and the output step depending on the subject-matter under examination and the objective of the examination, namely one or more of the steps b) qualification of at least one cell for transcriptome analysis by morphological or morphometric selection, c) storage of the cells in a frozen state, d) storage of the cells at 2-8° C., e) protease digestion of a tissue to generate individual cells.

In the context of these aspects of use of the present invention, it is particularly advantageous to provide the solution already in a sample tube or blood collection tube or syringe or another suitable vessel, such as the blood collection syringe. In particular, this allows the body fluid to be mixed directly with the solution according to the present invention after collection, so that falsification of the analysis, for example due to enzymatic activities, can be counteracted. Corresponding sample tubes or blood collection tubes or syringes or other reaction vessels containing the solution according to the present invention also represent further subject-matters of the present invention.

In this regard it is again particularly preferable to present the solution according to the present invention in concentrated form in order to avoid strong dilution of the sample. As an alternative to providing the solution in sample tubes or the like, mixing can also be carried out immediately after the blood sample or body fluid has been collected.

The sample tubes, blood collection tubes or syringes and other vessels containing the solution according to the present invention can be used in the context of the uses according to the present invention. However, like the solution according to the invention itself, these subject-matters are also suitable for carrying out other methods, such as analyses of cell-free DNA/RNA. The Use for such purposes is also encompassed by the present invention.

All the explanations given above for the solution according to the present invention shall also apply to the use of this solution according to the present invention as well as the prefabricated test tubes or blood collection tubes containing the solution according to the present invention.

The following examples, in connection with the figures, further explain the invention.

FIG. 1 shows fixed blood, as described in embodiment example 1, to which cultured tumor cells have been added and which has been examined by flow cytometry after 24 hours. The tumor cell population is clearly identifiable.

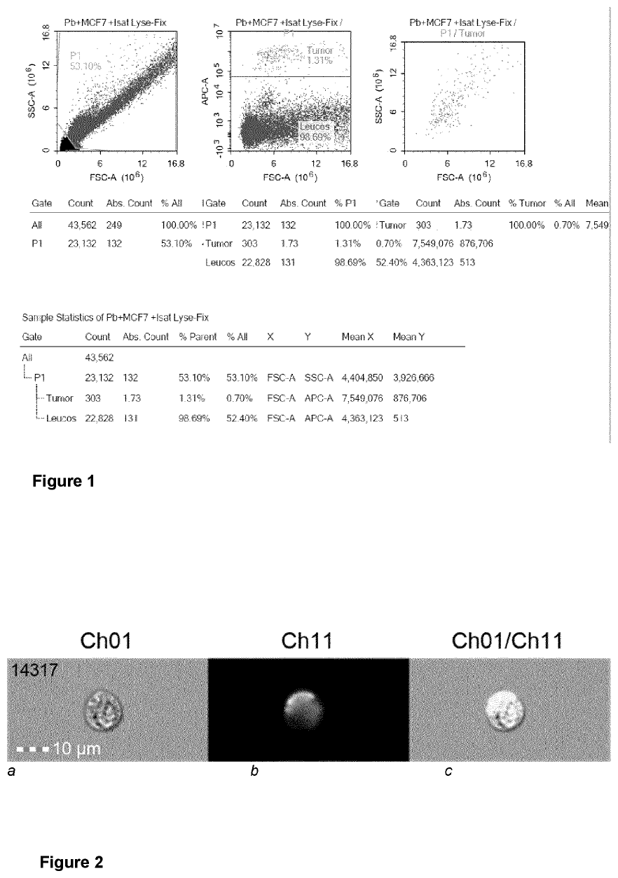

FIG. 2 shows an exemplary single cell analysis. Cultured tumor cells were added to fixed blood as described in embodiment example 1 and was labelled with an antibody against EpCam after 40 hours and examined by flow cytometry in the image stream. The cell morphology is preserved (2a). The fluorescence staining by the antibody is shown in 2b and the overlay of morphology and fluorescence is shown in 2C.

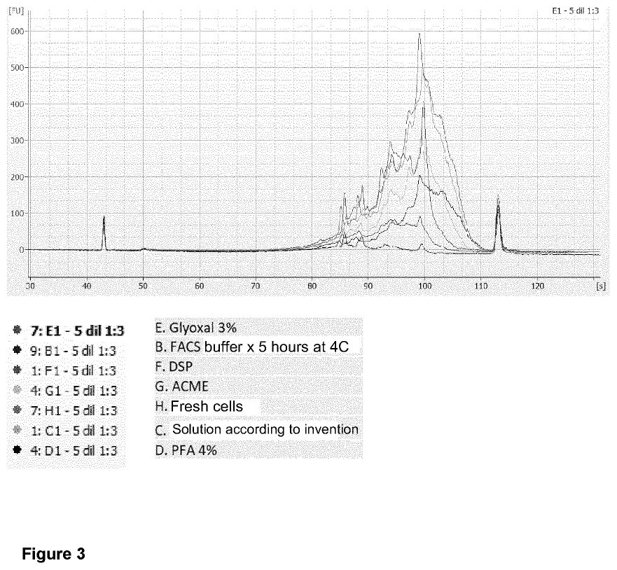

FIG. 3 shows a comparison of the integrity of the transcriptome after application of different cell fixation methods using an electrophoretic cDNA analysis after treatment of cells as described in embodiment 2. Cells treated with the solution according to the invention resemble fresh, untreated cells in their expression state.

### EXAMPLE 1

In this embodiment example, a solution according to the present invention having the following composition was used:

In this example, the solution according to the present invention is composed as follows:

23 mM glucose, 53 mM imidazole, 11 mM Hepes, 5 mM aspartic acid, 40 mM glutamic acid, 12 mM proline, 24 mM serine, 3.5 mM threonine, 32 mM alanine, 56 mM glycine, 10 mM MgSO4, 0.09% sodium azide, 150 mM acetic acid, HCl add pH 2.2

The solution has a pH of 2.2 at a temperature of 20° C. 2 ml of this solution was placed in a blood collection syringe. The syringe prepared in this way is used to take 8 ml of blood from a test subject and simultaneously prepare the mixture according to the present invention. The blood fixed in this way can be stored at 4° C. to 20° C. in order to subsequently analyze the mixture for the presence of CTC by microscopic or flow cytometric analysis, for example within 24 hours. Any CTC that may be present can also be isolated directly, e.g. via the affinity of surface markers to antibodies.

### EXAMPLE 2

HEK293 cells from cell culture were detached from the substrate using a trypsin solution and harvested. The cells were incubated with a live/dead dye and subsequently living cells were isolated. The cells were then incubated in an excess of different solutions, namely a FACS buffer, (Sheath fluid, BDBiosciences), a solution according to the present invention, a 4% paraformaldehyde PFA solution, a 3% glyoxal solution, a DSP solution (dithio-bis(succinimidyl propionate, a reversible crosslinker) or an ACME solution (ACetic-MEthanol: water, methanol, glacial acetic acid, glycerol in a ratio of 13:3:2:2) for 5 hours at 4° C. until further analysis. The cells were then lysed and mRNA was isolated. For comparison, fresh cells were lysed directly after harvesting.

In this example, the solution according to the present invention is composed as follows:

23 mM glucose, 53 mM imidazole, 11 mM Hepes, 5 mM aspartic acid, 40 mM glutamic acid, 12 mM proline, 24 mM serine, 3.5 mM threonine, 32 mM alanine, 56 mM glycine, 0.09% sodium azide, acetic acid (15 mM) add pH 5.3.

By reverse transcription cDNA for the entire cellular mRNA of the differently treated cells was generated and sequenced.

The comparison of the cDNA for the different samples showed that an extremely high cDNA integrity of the cells stored in the solution according to the present invention is observed, whereas for other storage solutions or buffers a clearly unfavorable preservation of the cellular transcriptome is ensured. FIG. 3 shows the results in graphical form, with the cDNA profile having the highest peak representing the result for freshly harvested cells. The cDNA profile with the second highest peak represents the solution according to the present invention and is very similar to the cDNA profile of fresh cells and most similar in comparison, while the profiles of other solutions deviate very strongly or do not resemble the profile of fresh cells at all.

1. Use of a solution for the conservation, preservation or/and fixation of at least one eukaryotic cell, in particular of tumor cells and circulating tumor cells, in a blood sample or another body fluid, or of cells from cell culture or of cells in tissue samples,
characterized in that
it is an aqueous solution which
a) contains a cell-permeable proton carrier, wherein the proton carrier is selected from C₂-C₅ carboxylic acids or a mixture of such carboxylic acids, or other acids which are membrane-permeable in protonated form and is present as an acid/base system, and forms a pH value in the acidic range.
b) is free of such amounts of chaotropic substances, alcohols or detergents, which destroy morphological integrity of the cells,
c) maintains the morphological integrity of the cells, and
d) has a pH value that generates an intracellular pH of the cells of less than 6.

2. The use according to claim 1,
characterized in that
acidic acid/acetate system is present in the solution.

3. The use according to claim 2,
characterized in that the acid/base system is present in a concentration of 1 mM to 100 mM, preferably 5 mM to 50 mM and particularly preferably 10 mM to 20 mM.

4. The use according to claim 3,
characterized in that the acid/base system is configured as a 2-20-fold multiple concentrate, particularly preferably as a 5-fold concentrate.

5. The use according to claim 1,
characterized in that the solution additionally contains a strong acid, preferably hydrochloric acid or another mineral acid, to adjust the intracellular pH value.

6. The use according to claim 1,
characterized in that the pH value of the solution after mixing with cells or other biological material is an acidic pH, preferably between 4 and 6, particularly preferably between 4.5 and 5.5 and most preferably 5.3, in each case measured at 20° C.

7. The use according to claim 1,
characterized in that the solution contains further buffer substances or proton carriers.

8. The use according to claim 1,
characterized in that the solution contains imidazole or/and DMSO.

9. The use according to claim 1,
characterized in that the solution comprises at least one sugar, which is preferably selected from the group of monosaccharides or disaccharides, or/and at least one amino acid, which is preferably selected from the group comprising glycine, alanine, proline, serine, threonine, glutamic acid, aspartic acid.

10. The use according to claim 9,
characterized in that said one or more amino acids are present in a concentration of 0.1 M to 1 M in total or in each case, preferably in a total concentration of 100 mM to 300 mM.

11. The use according to claim 1,
characterized in that the solution additionally contains a preservative active ingredient from the group of formaldehyde donors, in particular diazolidinyl urea or imidazolidinyl urea.

12. The use according to claim 1,
characterized in that the osmolarity of the solution does not exceed 1 osmol, preferably is below 500 mosmol, and in particular forms an isotonic environment for the eukaryotic cell or cells to be preserved.

13. The use according to claim 1,
characterized in that the solution contains complexing agents or coagulation-preventing agents or inhibiting antibodies.

14. The use according to claim 13,
characterized in that the coagulation-preventing agent is selected from MgSO₄, dabigatran, plasminogen activator or any mixtures of these substances.

15. The use of a solution according to claim 1, wherein the solution is mixed with the blood sample or body fluid, the cell culture or tissue sample is mixed directly after removal or/and the solution is provided in a sample tube, a blood collection tube or a blood collection syringe, and the blood sample, body fluid, cell culture or tissue sample is added to this vessel, wherein the solution is preferably provided or added in concentrated form.

16. The use according to claim 1, wherein the cell or cells remain substantially intact and RNA from at least one cell is used for a transcriptome analysis.

17. The use according to claim 1, wherein a morphological analysis of at least one marker protein of the cell or cells is used to qualify at least one cell for the transcriptome analysis.

18. The use according to claim 1, comprising an input step a) mixing cells in liquid or tissue with the solution according to any one of claims 1 to 14 and an output step f) molecular analysis of the transcriptome of at least one cell.

19. The use according to claim 18, comprising one or more intermediate steps selected from b) quantification of at least one cell for transcriptome analysis by morphological or morphometric selection, c) storage of the cells in a frozen state, d) storage of the cells at 2 to 8° C., e) protease digestion of a tissue to generate individual cells.

20. A sample tube, blood collection syringe or blood collection tube, which are characterized in that they contain a solution for the conservation, preservation or/and fixation of at least one eukaryotic cell, in particular of tumor cells and circulating tumor cells, in a blood sample or another body fluid, or of cells from cell culture or of cells in tissue samples, which
a) contains a cell-permeable proton carrier, wherein the proton carrier is selected from C₂-C₅ carboxylic acids or a mixture of such carboxylic acids, or other acids which are membrane-permeable in protonated form and is present as an acid/base system, and forms a pH value in the acidic range.
b) is free of such amounts of chaotropic substances, alcohols or detergents, which destroy the morphological integrity of the cells,
c) maintains the morphological integrity of the cells, and
d) has a pH value of less than 6 that generates an intracellular pH of the cells of less than 6.
